(19) United States
(12) Patent Application Publication
(10) Pub. No.: US 2008/0200923 A1
(43) Pub. Date: Aug. 21, 2008

(54) Insertion tool for ocular implant and method for using same

(76) Inventors: Richard Beckman, New York, NY (US); Dana Cote, Boxford, MA (US); Mark Bowen, Stow, MA (US); Eric A. Bene, Lynn, MA (US); Michael McGraw, Brighton, MA (US)

Correspondence Address: David W. Highet, VP & Chief IP Counsel;Becton, Dickinson and Company, (Roylance Abrams Berdo & Goodman), 1 Becton Drive, MC 110, Franklin Lakes, NJ, 07417-1880, US

(21) Appl. No.: 12/007,259

(22) Filed: Jan. 8, 2008

Related U.S. Application Data
(60) Provisional application No. 60/879,338, filed on Jan. 9, 2007.

(51) Int. Cl. A61F 9/007

(52) U.S. Cl. 606/108; 128/898; 604/8

(57) ABSTRACT

An insertion tool and method are provided for implanting an ophthalmic shunt having a foot, a head and a body connecting the foot and head. The insertion tool has a first arm having a proximal end and a distal end and a second arm having a proximal end and a distal end. The second arm is movable with respect to the first arm between an open position and a closed position. A means to grip a shunt is disposed on the distal ends of the first and second arms. In use, the insertion tool is employed to selectively grip the shunt for placement in the tissue of the eye and, as desired, to orient the shunt after placement.

### CROSS-REFERENCE TO RELATED APPLICATION

This application claims the benefit of U.S. Provisional Application No. 60/879,338, filed Jan. 9, 2007, in the U.S. Patent and Trademark Office, the disclosure of which is incorporated herein by reference in its entirety.

### FIELD OF THE INVENTION

The present invention relates to devices and methods for use with ocular and non-ocular implants. More particularly, aspects of the present invention relate to insertion tools and methods for the controlled insertion of an ophthalmic shunt into an eye to relieve intraocular pressure.

### BACKGROUND

Glaucoma, caused by optic nerve cell degeneration, is the second leading cause of preventable blindness in the world today. A major symptom of glaucoma is a high intraocular pressure, or “IOP”, which is caused by the trabecular meshwork failing to drain enough aqueous humor from within the eye. Glaucoma therapy is directed at protecting the optic nerve and preserving visual function by attempting to lower IOP using various methods, such as through the use of drugs or surgery, including surgical methods such as trabeculectomy.

Trabeculectomy is an invasive surgical procedure in which no device or implant is used. Typically, surgery is performed to puncture, or reshape, the trabecular meshwork, by surgically creating a channel opening the sinus venosus. Another surgical technique used involves the use of implants within the eye, such as stents or shunts, which are typically quite large and are implanted during a surgically invasive procedure. These implants work to relieve internal eye pressure by permitting aqueous humor to flow from the anterior chamber, through the sclera, and into a conjunctive bleb over the sclera. These procedures are very labor intensive for the surgeons and can be subject to failure due to scarring and cyst formations.

Another solution to the problems encountered involves using a transcorneal shunt as shown in place in FIG. 1. A transcorneal shunt reduces intraocular pressure in the eye by shunting aqueous humor from the anterior chamber of the eye, through the cornea, to the tear film. By draining aqueous humor through the cornea, the transcorneal shunt makes surgical implantation of the device less invasive and allows for quicker surgery than with other surgical options. Additional details of transcorneal shunts are described in U.S. Pat. No. 5,807,302 and in Published International Patent Application Nos. WO 01/50943 and WO 2005/117780, the entire content of each being incorporated herein by reference. In certain applications, the structure depicted in FIG. 1 may be employed in trans-scleral applications, as discussed more fully below.

The transcorneal shunt 10 of FIG. 1 is constructed having a first flange or head 12 at a proximal end to anchor the shunt 10 on the outside surface of the cornea 14, and a second flange or foot 16 at a distal end to anchor the shunt 10 on the inside surface 18 of the cornea. A body 20 that forms an internal conduit extends between the first and second flanges. The conduit can include a filter (not shown) designed to restrict bacteria from infiltrating into the eye through the shunt 10. The conduit and filter may be designed to control the flow rate of the aqueous humor from the anterior chamber of the eye to the outside surface of the cornea.

The transcorneal shunt 10 is inserted, or implanted, in the cornea through a small incision. The incision is sized to allow the foot 16 to be manipulated through the incision and yet prevent the head 12 and foot 16 from passing through once the shunt 10 is in place (thereby securing the shunt 10 in position). Due to the nature of this procedure, it is desirable to provide a device and a method for insertion that permits the surgeon to have precise control over the position of the shunt 10, visual access to the shunt 10 during insertion, and control over when the shunt 10 is released.

Attempts to develop shunt implantation tools include insertion tools that house the shunt in a tubular tip, and insert the shunt by a pressing motion against the surface of the cornea. Such insertion tools typically include a stiff tube and a plunger assembly, and the shunt is held within the tubular section of the assembly at the tip of the tool. When the tool is pressed down against the eye, the plunger pushes the shunt out of the tubular tip and into the cornea incision. Other types of known insertion devices are described in Published International Patent Application No. WO 2004/105659, the entire content of which is incorporated herein by reference.

A need exists for a tool for inserting a transcorneal shunt through the cornea of the eye that can gently grasp, but also securely hold the proximal end of the shunt without damage to the delicate shunt structure, such that the incision and shunt are not hidden by the tool so that the surgeon can easily view, manipulate and insert the shunt through the cornea.

### SUMMARY OF EMBODIMENTS OF THE INVENTION

An object of one aspect of the present invention is to address at least the above needs and to provide at least the advantages described below. Accordingly, an object of an aspect of this invention is to provide a device, and method for using the same, that may be used to insert an ophthalmic shunt into the eye to relieve IOP by draining the anterior chamber of the eye of aqueous humor.

An object of another aspect of the present invention is to provide a device, and method for using the same, that may be used to implant a transcorneal shunt through the cornea of the eye such that the shunt can be placed across the cornea to relieve IOP by draining the anterior chamber of the eye of aqueous humor.

An object of another aspect of the present invention is to provide a device for shunt implantation, which can gently, but also securely, hold the proximal end of a shunt, such that the surgeon can easily view an incision and exposed shunt portion, manipulate, and insert the shunt into the eye.

An object of another aspect of the present invention is to provide a device for shunt implantation, which can gently hold the proximal end of the shunt after the distal end has been inserted into the anterior chamber of the eye to provide user feedback regarding whether the distal flange has fully extended within the anterior chamber of the eye.

An object of another aspect of the present invention is to provide a device for shunt implantation, which can be easily fabricated from a minimum of parts.

An object of another aspect of the present invention is to provide a device for shunt implantation, which provides a surgeon with increased control over the release of the shunt.

An object of another aspect of the present invention is to provide a device for shunt implantation, which provides a surgeon with increased control over the insertion by using finger movements (i.e., micro movements) rather than wrist and hand movements (i.e., macro movements).

An object of another aspect of the present invention is to provide a device for shunt implantation, which provides a surgeon with increased visibility of the shunt and the incision site during implantation.

These and other objects are substantially achieved by providing a shunt insertion tool for implanting an ophthalmic shunt having a foot, a head, and a body connecting the foot and head. The insertion tool includes a first arm having a proximal end and a distal end, and a second arm having a proximal end and a distal end. The second arm is movable with respect to the first arm between an open position and a closed position. A means for gripping a shunt is disposed on the distal ends of the first and second arms.

A method is provided for treating glaucoma by creating an incision in the cornea. An insertion tool is employed to gently but securely grip a shunt. The edge of a flange on the shunt is displaced into the incision. The shunt is rotated, displacing the flange through the cornea. The insertion tool is disengaged from the shunt.

### BRIEF DESCRIPTION OF THE DRAWINGS

The above and other objects, features, and advantages of certain exemplary embodiments of the present invention will be more apparent from the following description taken in conjunction with the accompanying drawings, in which:

FIG. 1 is an enlarged cross-sectional view of a transcorneal shunt, or ocular implant, that may be inserted using an insertion tool in accordance with an embodiment of the present invention;

FIG. 2 is a bottom plan view of an insertion tool according to an exemplary embodiment of the present invention, in an open position;

FIG. 3 is a front view of the insertion tool shown in FIG. 2, in an open position;

FIG. 4 is a top plan view of the insertion tool shown in FIG. 2, in a closed position;

FIG. 5 is a front view of the insertion tool shown in FIG. 2, in a closed position;

FIG. 6 is an enlarged front view of a first area indicated in FIG. 5;

FIG. 7 is an enlarged top view of a first area indicated in FIG. 2;

FIG. 8 is an enlarged front view of the first area indicated in FIG. 2;

FIG. 9 is an enlarged top view of a second area indicated in FIG. 2;

FIG. 10 is an enlarged side view of the second area indicated in FIG. 2;

FIG. 11 is a perspective view of an insertion tool according to another exemplary embodiment of the present invention, in an open position;

FIG. 12 is an enlarged perspective view of the tip of the insertion tool illustrated in FIG. 11, with a shunt in place;

FIG. 13 is a sectional view of the tip of the insertion tool illustrated in FIG. 11, with a shunt in place and the tool in a closed position;

FIG. 14 is a sectional view of the tip of the insertion tool illustrated in FIG. 11, with a shunt in place and the tool in an open position;

FIG. 15 is a perspective view of an alternative configuration for the arms of the insertion tools shown in FIG. 1, according to another exemplary embodiment of the present invention, in a closed position;

FIG. 16 is a front view of an insertion tool according to another exemplary embodiment of the present invention, in a closed position;

FIG. 17 is an exploded, perspective view of the insertion tool illustrated in FIG. 16;

FIG. 18 is an enlarged view of a latching mechanism of the insertion tool illustrated in FIG. 16, in a closed position;

FIG. 19 is an enlarged view of the latching mechanism of the insertion tool illustrated in FIG. 16, in an open position;

FIG. 20 is a front view of an insertion tool according to another exemplary embodiment of the present invention, in a closed position;

FIG. 21 is an exploded, perspective view of the insertion tool illustrated in FIG. 16;

FIG. 22 is a sectional view of an enclosure with a sliding cam mechanism for use with the insertion tool illustrated in FIG. 2, in an open position;

FIG. 23 is a sectional view of the enclosure for an insertion tool illustrated in FIG. 22, in a closed position;

FIG. 24 is a sectional view of a variation of the sliding cam mechanism of FIG. 22;

FIG. 25 is a perspective view of the tip of an insertion tool according to another exemplary embodiment of the invention;

FIG. 26 is a schematic diagram of a shunt ejection mechanism for use with the insertion tool illustrated in FIG. 25;

FIG. 26A is a schematic diagram of an alternative shunt ejection mechanism for use with the insertion tool illustrated in FIG. 25;

FIG. 27 is a perspective view of a cap for retaining a shunt on an insertion tool;

FIG. 28 is a schematic side view of the cap illustrated in FIG. 27 installed on an insertion tool, with the insertion tool in an open position;

FIG. 29 is a schematic side view of the cap illustrated in FIG. 27 installed on an insertion tool, with the insertion tool in a closed position;

FIG. 30 is a side view of an elastomeric cap for retaining a shunt on an insertion tool;

FIG. 31 is a perspective view of the elastomeric cap shown in FIG. 30;

FIG. 32 is a side view of an elastomeric cap for retaining a shunt on an insertion tool;

FIG. 33 is a sectional view of the elastomeric cap shown in FIG. 32;

FIGS. 33A and 33B are views of another cap for retaining a shunt on an insertion tool;

FIG. 34 is a perspective view of a package for an insertion tool;

FIG. 35 is a top view of another package for an insertion tool, in a closed state;

FIG. 36 is a top view of the package shown in FIG. 35, in an open state;

FIG. 37 is a bottom plan view of a device for holding a shunt according to an exemplary embodiment of the invention;

FIG. 38 is a front view of the device shown in FIG. 37;

FIG. 39 is a top plan view of the device shown in FIG. 37;

FIG. 40 is a right side view of the device shown in FIG. 37;

FIG. 41 is a sectional view of the device shown in FIG. 37;

FIG. 42 is a sectional view of the device shown in FIG. 37;

FIG. 43 is an illustration of a slot-shaped aperture for use with the holding device of FIG. 37;

FIG. 44 is an illustration of an aperture formed by three petal-shapes for use with the holding device of FIG. 37;

FIG. 45 is an illustration of an aperture formed by a slot and a circle for use with the holding device of FIG. 37; and

FIGS. 46-51 are illustrations of a method of using the holding device of FIG. 37;

FIG. 52 is a perspective view of a device for holding a shunt according to an another exemplary embodiment of the invention;

FIG. 53 is a perspective view of a device for holding a shunt according to an another exemplary embodiment of the invention; and

FIG. 54 is a perspective view of a device for holding a shunt according to an exemplary embodiment of the invention.

Throughout the drawings, the same reference numerals will be understood to refer to the same elements, features, and structures.

### DETAILED DESCRIPTION OF EXEMPLARY EMBODIMENTS

The transcorneal shunt (hereinafter “shunt”) has been developed to reduce intraocular pressure (IOP) in the eye by shunting aqueous humor from the anterior chamber of the eye, through the cornea, and to the tear film. To do so, the shunt must be implanted through a small incision and into the cornea of the eye, actually extending between the inner and outer surface of the cornea. The shunt, however, is very small and light, requiring particular care during such insertion procedures. The embodiments of the present invention described below enable a surgeon to gently grasp the shunt with an implantation tool and hold the shunt in position until manually released. The shunt remains visible to the surgeon, allowing greater control and precision during implantation.

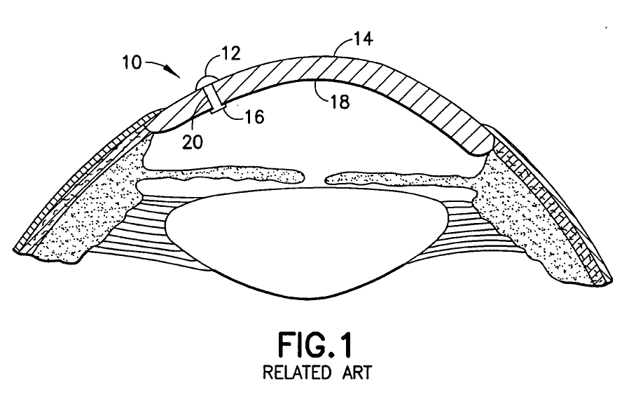

As seen in FIG. 1, the shunt 10 has a first flange 12 at a proximal end to anchor the shunt on the outside surface of the cornea 14, and a second flange 16 at a distal end to anchor the shunt 10 on the inside surface 18 of the cornea 14. A body 20 extends between the first and second flanges 12, 16, and includes an internal conduit, which can include a filter (not shown) designed to restrict bacteria from infiltrating into the eye through the shunt. For convenience, the first flange 12 of the shunt 10 will be referred to as a “head,” and the second flange 16 will be referred to as a “foot.” As depicted in FIG. 1, the shunt is made of a hydrogel and has been rehydrated to seat snugly in the cornea.

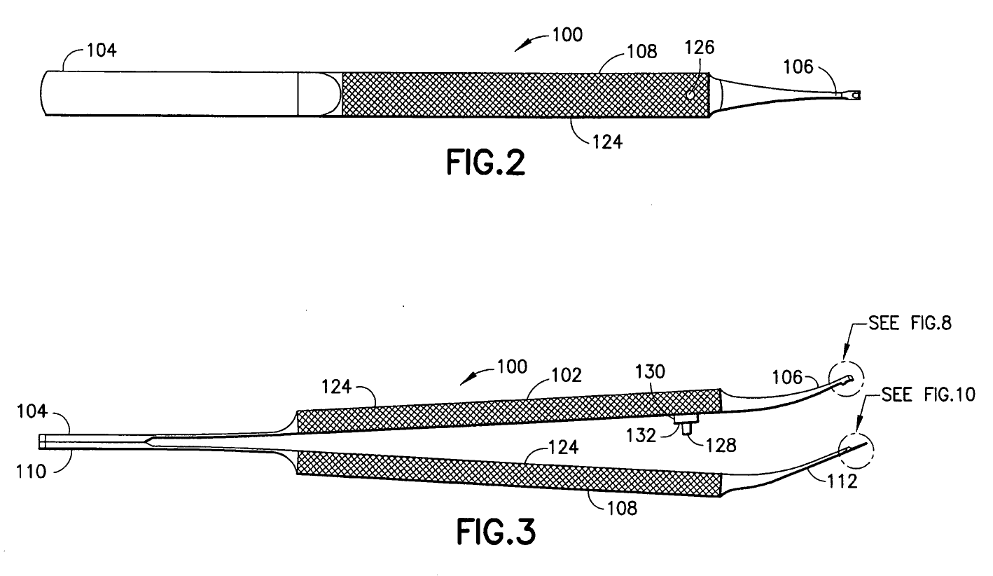

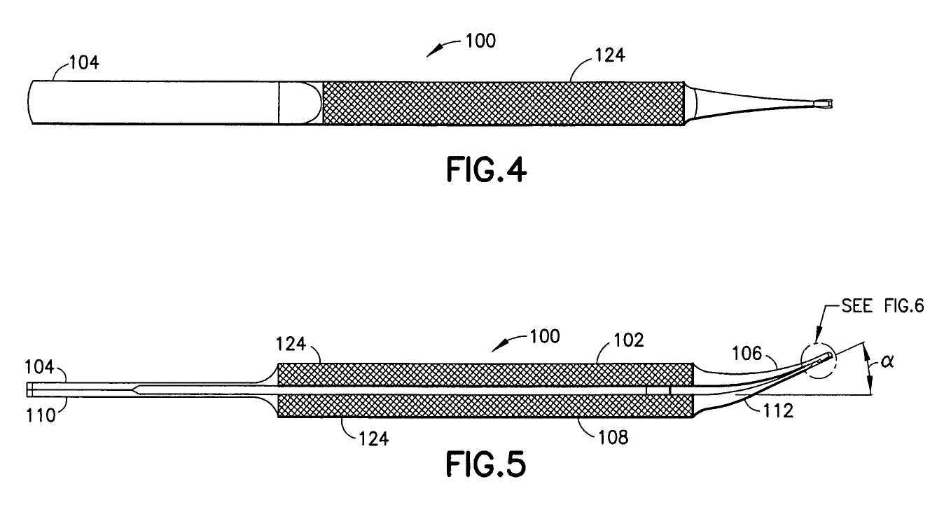

FIGS. 2-10 illustrate a tool for inserting a shunt according to a first exemplary embodiment of the present invention. The insertion tool 100 includes a first arm 102 having a proximal end 104 and a distal end 106, and a second arm 108 having a proximal end 110 and a distal end 112. The proximal ends 104, 110 of the first and second arms 102, 108 are joined so that the arms are pivotable between an open position (FIGS. 2-3) and a closed position (FIGS. 4-5). In the illustrated embodiment, in the open position, the distal ends 106, 112 of the first and second arms 102, 108 are separated by about 0.31″ (7.87 mm). The first and second arms 102, 108 extend generally longitudinally to define a longitudinal axis, and the arms pivot in a direction substantially perpendicular to the longitudinal axis of the insertion tool 100. That is, the arms pivot in a vertical direction, with reference to FIG. 3. In the illustrated embodiment, the arms are biased toward the open position. That is, when no force is applied to the arms, the arms are in the position shown in FIG. 3.

The first and second arms 102, 108 may be formed of any suitable material, such as stainless steel or a polymer, such as a polycarbonate. The material preferably has sufficient strength so that it can be sterilized, and different materials may be combined. The arms may be formed as separate members, and then joined together (such as by brazing, welding, riveting, insert molding, or any other technique known to those skilled in the relevant art), or may be formed as a unitary member (such as by EDM or wire machining). The insertion tool 100 may be a disposable device, or may be reusable.

When closed, the distal ends 106, 112 of the first and second arms 102, 108 are at angle α with respect to the longitudinal axis of the insertion tool 100. The angle α is chosen to minimize any obstruction of a surgeon's vision when using the insertion tool, and to minimize any interference with a patient's anatomy (nose, forehead, etc.). In the illustrated embodiment, the angle α is approximately 25°.

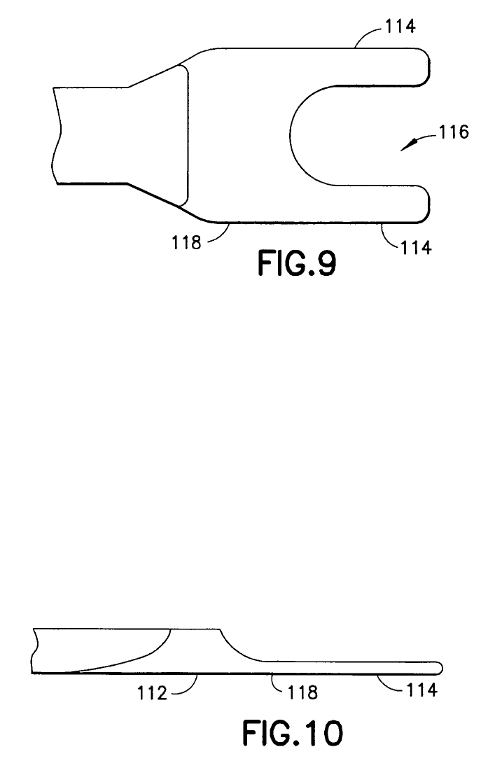

As best seen in FIGS. 9 and 10, the distal end 112 of the second arm 108 is formed as a thin, spatula blade 118 with two prongs 114 that form a slot 116. The size of the slot 116 is chosen so that it is large enough to accommodate the body 20 of a shunt, but small enough that the proximal and distal flanges of the shunt cannot pass through the slot 116. In the illustrated embodiment, the slot 116 is 0.033″ (0.84 mm) wide. Preferably, the width of the blade 118 is approximately equal to the diameter of a head of a shunt, and is thin enough to allow full insertion of the foot of a shunt through the cornea. In the illustrated embodiment, the blade 118 is 0.060″ (1.5 mm) wide and 0.005″ (0.13 mm) thick. The slot 116 is substantially parallel to the longitudinal axis of the insertion tool 100. In the illustrated embodiment, the slot 116 is 0.050″ (1.27 mm) long. In an illustrative embodiment, the shunt is made of a hydratable material, and the shaft of the shunt has a diameter of approximately 0.029″ (0.74 mm) in a dehydrated state so that there is a small gap between the edges of the slot and the shaft of the shunt. This allows the shunt to expand when it is hydrated (i.e. exposed to moisture), such as during a sterilization procedure, without damaging the shunt. For example, the shunt may have a diameter of approximately 0.034″ (0.86 mm), which is slightly larger than the width of the slot 116, when it is hydrated.

As seen in FIGS. 7-8, the distal end 106 of the first arm 102 of the insertion tool 100 has a gripping surface 120 for grasping the head of a shunt. The gripping surface 120 is aligned with and opposes the blade 118 disposed on the second arm 108 of the insertion tool 100. Preferably, the gripping surface 120 is a concave surface that accommodates the head of a shunt.

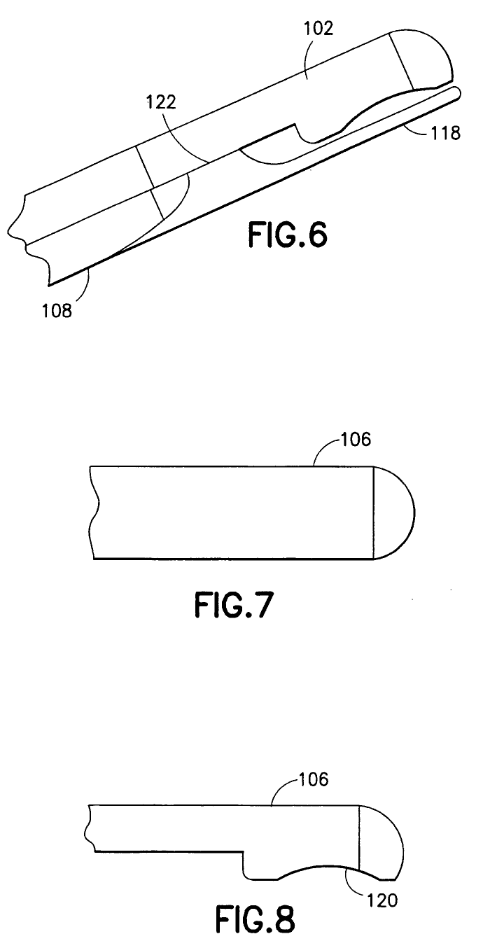

As seen in FIG. 6, the distal end 108 of the second arm 108 may have an abutment surface 122 that contacts the first arm 102 when the first and second arms 102, 108 are closed. The abutment surface 122 maintains a gap between the first and second arms, and maintains a small gap between the head of a shunt inserted into slot 116 of the second arm 108 and the gripping surface 120. This allows a dehydrated shunt to be shipped and stored in the tool without damaging the shunt and also provides clearance so that the shunt may expand when it is exposed to moisture without damaging the shunt. Furthermore, when the insertion tool is used to implant a shunt, the shunt absorbs moisture from the tear film of a patient. Thus, the shunt is in a hydrated state, and is held stably while the tool is used by a surgeon.

As shown in the illustrated embodiment, the first and second arms 102, 108 of the insertion tool 100 preferably have knurled or dimpled surfaces 124 (e.g., a fine diamond knurl) for gripping. The knurled surfaces form a handle to allow a surgeon to firmly grasp the tool without slippage. The handle of the insertion tool 100 may be round, similar to other ophthalmic instruments such as round tying forceps or knives, so that the tool may be rotated by a surgeon. The overall length of the insertion tool 100 is such that it may fit into a surgeon's hand, approximately 5″ to 7″ (127 mm to 178 mm).

To maintain alignment of the distal ends 106, 112 of the first and second arms 102, 108, an alignment aperture 126 is provided in one of the arms (in the illustrated embodiment, the first arm), and an alignment pin 128 is provided in the other arm. The alignment pin 128 engages the alignment aperture 126 to maintain alignment of the first and second arms 102, 108. A limit pin 130 may be provided on one of the arms. The limit pin forms an abutment surface 132 which engages the other arm to maintain a desired clearance between the arms, as seen in FIG. 5. The limit pin 130 may be coaxial with the alignment pin 128, as illustrated.

The process of implanting a shunt using the insertion tool of FIGS. 2-10 will now be described. Initially, prior to implanting a shunt, the shunt and the insertion tool 100 should be sterilized to eliminate bacteria, viruses, fungi and the like. To do so, the insertion tool 100 and the shunt may be autoclaved, either together (i.e., while the shunt is inserted into the tool) or, preferably, separately. After the shunt and insertion tool have been sterilized and the shunt has been loaded onto the insertion tool 100, the shunt is ready for implantation.

Before initiating the surgery, pre-operative pachymetry is performed to determine the thickness of the cornea. Using these measurements, the surgeon determines to optimal location for placement of the shunt in the cornea, as well as the appropriate size of the shunt to be inserted. Specifically, the length of the shunt between the flanges is selected based on the thickness of the cornea at the point of insertion when the shunt is re-hydrated in the eye. A retro-bulbar injection or a topical anesthetic may be applied to anesthetize the eye. The eye may then be decompressed by withdrawing fluid from the anterior chamber. The patient is then prepped and draped. Paracentesis is performed and the eye re-inflated with a saline solution or a suitable viscoelastic material. At this point, an incision is made through the cornea at a length which is sufficient to allow insertion of the chosen shunt size. The incision may be made by any suitable tool known to those skilled in the relevant art. As currently envisioned, the incision is parallel to the limbus and is normal to the surface of the cornea. It certain applications, however, it may be desirable to address the corneal surface at another angle (which may create a self-sealing incision). It should be understood that the above described method is only an example of one suitable method of inserting a shunt, and individual surgeons may prefer slightly different methods.

With the incision made, the surgeon uses the insertion tool 100 to implant the shunt. The insertion tool 100 is held in a closed position so that the shunt is captured securely in the slot 116 of the insertion tool 100. The surgeon then rotates the insertion tool 100 so that one edge of the foot of the shunt contacts the surface of the eye, preferably near the incision. The surgeon may move the shunt along the surface of the eye until the edge of the foot engages the incision. During this movement, the shunt is gripped laterally (rather than axially), so that the tool minimizes interference with the surgeon's vision.

Once the shunt is at the proper location, the surgeon may implant the shunt by rotating and displacing the insertion tool 100 (this movement is induced by “rolling” the handle of the tool between the surgeon's fingers) so that the remainder of the foot of the shunt is forced through the incision, thereby placing the foot of the shunt under the inner surface of the cornea. This procedure is similar to inserting a button into a button hole. If necessary, the surgeon may apply additional pressure to the head of the shunt to ensure that the foot of the shunt is passed completely through the cornea. Once the surgeon is satisfied with the placement of the shunt, the surgeon may gradually release the first arm 102, which is naturally biased toward an open position, in a controlled manner, so that the insertion tool 100 is opened. The surgeon may then withdraw the blade 118 from underneath the head of the shunt by moving the insertion tool 100 backward, and the shunt is implanted.

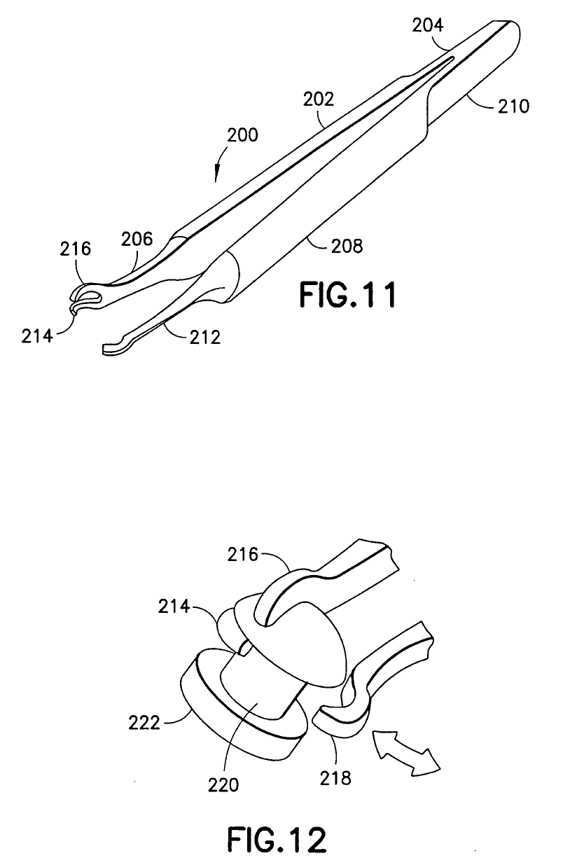

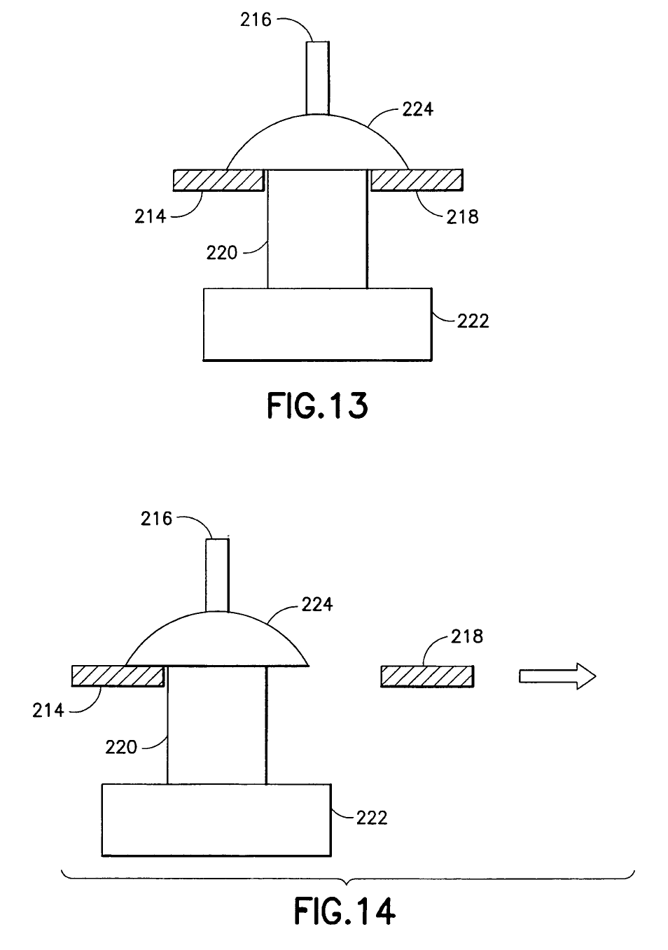

FIGS. 11-14 illustrate a tool for inserting a shunt according to a second exemplary embodiment of the present invention. As seen in FIGS. 11-14, the insertion tool 200 includes a first arm 202 having a proximal end 204 and a distal end 206, and a second arm 208 having a proximal end 210 and a distal end 212. The proximal ends 204, 210 of the first and second arms 202, 208 are joined so that the arms are pivotable between an open position (FIG. 14) and a closed position (FIG. 13). The arms pivot in a generally lateral direction, and, in the illustrated embodiment, the arms are biased toward an open position.

The distal end 206 of the first arm 202 of the insertion tool 200 includes a first side jaw 214 and an upper prong 216. The first side jaw 214 is curved to create a recess for receiving the body 220 of a shunt 222. The upper prong 216 is also curved to create a recess for receiving the head 224 of a shunt 222. The first side jaw 214 and the upper prong 216 are fixed with respect to one another so that they move together.

The distal end 212 of the second arm 208 of the insertion tool 200 includes a second side jaw 218. The second side jaw 218 is curved to create a recess for receiving the body 220 of a shunt 222. As seen in FIG. 13, when the second side jaw 218 is closed with respect to first side jaw 214, the body 220 of the shunt 222 is received in the recesses of the first and second side jaws 214, 218. On the other hand, when the second side jaw 218 is open with respect to the first side jaw 214, the body 220 of the shunt 222 is released and may be removed from the insertion tool 100.

Although not illustrated, the insertion tool 200 of this embodiment may have an abutment surface, an alignment pin, and a limit pin as described above with respect to the first embodiment of the invention to maintain the proper clearances and alignment between the first and second arms 202, 208 of the insertion tool 200.

The operation of the second embodiment of the insertion tool 200 is substantially the same as the operation of the first embodiment of the insertion tool 100, except that the shunt is released by moving the arms laterally, instead of vertically. Accordingly, a detailed description of the operation will not be repeated for clarity and conciseness.

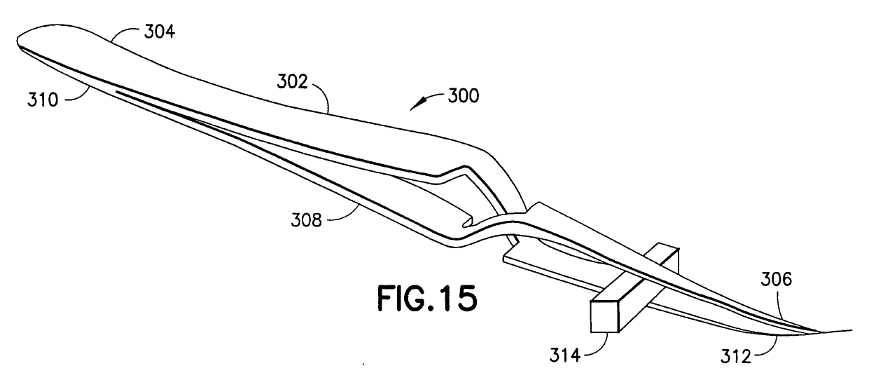

FIG. 15 illustrates an insertion tool 300 in accordance with another exemplary embodiment of the present invention. The insertion tool 300 includes a first arm 302 having a proximal end 304 and a distal end 306, and a second arm 308 having a proximal end 310 and a distal end 312. The proximal ends 304, 310 of the first and second arms 302, 308 are joined so that the arms are pivotable between an open position (not illustrated) and a closed position (FIG 15). The first and second arms 302, 308 of the insertion tool cross over one another. When the arms cross over in this manner, the insertion tool 300 is naturally biased into a closed position. Thus, in contrast to the first and second embodiments described above, a surgeon must squeeze the insertion tool 300 to open the tool to release a shunt. The remainder of the tool may be constructed as described above with respect to the first and second embodiments of the invention, and the remaining operations of the shunt are substantially the same as described above.

By constructing the insertion tool 300 in this manner, the insertion tool 300 may be shipped, sterilized, and handled with little risk of dropping the shunt. A removable stop 314 may be inserted between the arms 302, 308 of the insertion tool 300. The removable stop 314 prevents the arms 302, 308 from completely closing to allow the insertion tool 300 to hold the shunt loosely during storage, sterilization, and shipping.

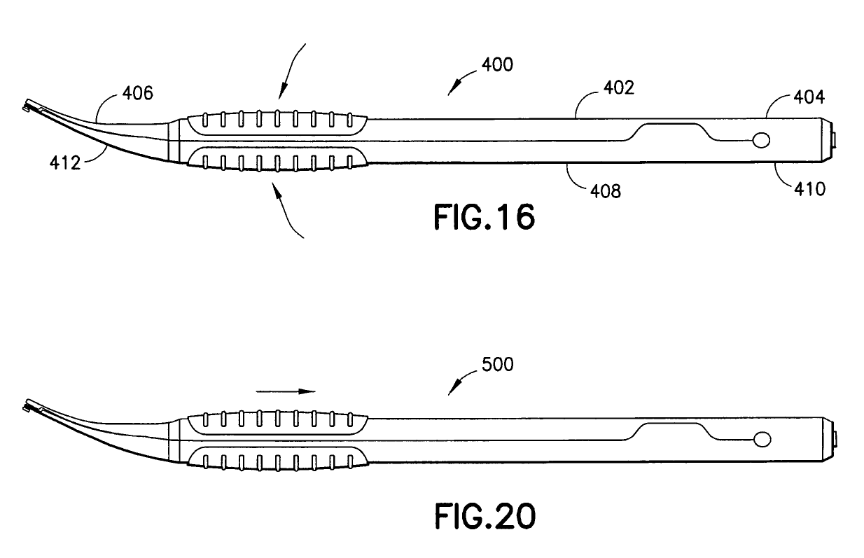

FIGS. 16-19 illustrate an insertion tool 400 in accordance with another exemplary embodiment of the present invention. The insertion tool 400 includes a first arm 402 having a proximal end 404 and a distal end 406, and a second arm 408 having a proximal end 410 and a distal end 412. The proximal ends 404, 410 of the first and second arms 402, 408 are joined so that the arms are pivotable between an open position and a closed position. In the illustrated embodiment, the arms 402, 408 are biased toward an open position. The arms 402, 408 pivot in a generally vertical direction. As will be described in further detail below, a “squeeze to release” latching mechanism holds the arms 402, 408 in a closed position until a user squeezes the mechanism to release the latch so that the arms 402, 408 may be opened.

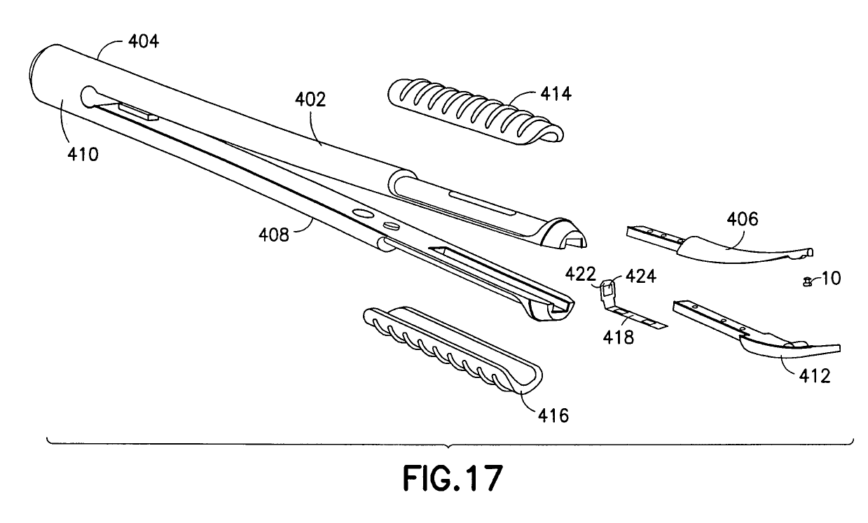

As seen in FIG. 17, the first and second arms 402, 408 are each formed in two members. The proximal ends 404, 410 of the arms 402, 408 are formed of injection molded plastic. In the illustrated embodiment, the proximal ends 404, 410 of the arms 402, 408 are formed as a single, unitary member. The arms may, however, be formed as separate members, and then joined together by any suitable method, such as adhesive or ultrasonic welding, or over molding.

The distal ends 406, 412 of the first and second arms 402, 408 are formed as separate members. Preferably, the members are formed of stamped sheet metal. Other methods known to those skilled in the relevant art, such as powder metallurgy, machining, metal injection molding, casting, and so on, may also be used to manufacture the members, and alternative materials, such as plastics, may also be used. In the illustrated embodiment, the distal ends 406, 412 of the first and second arms 402, 408 are configured substantially the same as described above with respect to the first embodiment of the invention. The arms may also be configured the same as described above with respect to the second embodiment of the invention. The distal ends 406, 412 of the first and second arms 402, 408 are preferably molded together with the proximal ends of the first and second arms by using insert molding techniques. Alternatively, they may be joined to the molded arms using any conventional technique.

First and second handle members 414, 416 are attached to the first and second arms 402, 408, respectively. The handle members may be formed by injection molding plastic, and may be joined to the arms by any suitable method, such as by adhesives or welding.

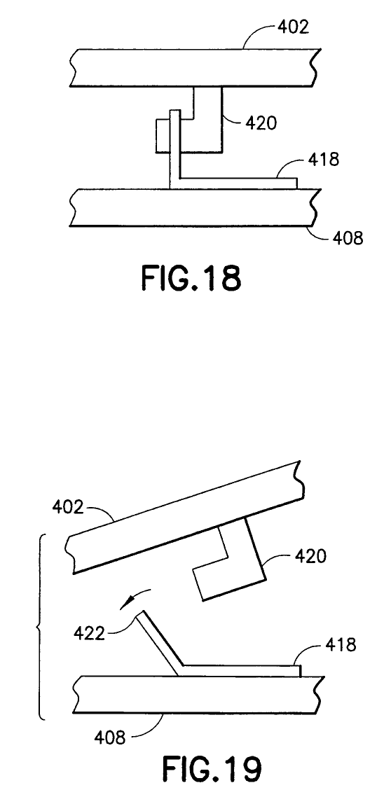

A latching mechanism is provided to hold the arms in a closed position. The latching mechanism includes a latching plate 418 and a hooking member 420. The latching plate 418 is formed of an elastic material, such as spring steel. A first end 422 of the latching plate 418 is biased toward an open position, as indicated by the arrow in FIG. 19. To use the latching mechanism, the arms 402, 408 are placed into a closed position. A tool such as a thin blade is inserted into the gap between the first and second arms 402, 408, and used to move the first end 422 of the latching plate 418 from the position shown in FIG. 19 to the position shown in FIG. 18. In this position, an aperture 424 in the latching plate 418 is placed over the end of the hooking member 420. The arms are released, and the tool is removed. At this time, the first and second arms 402, 408 are naturally biased outward, and the latching plate 418 is fixed with respect to the hooking member 420 by frictional forces between the latching plate 418 and the hooking member 420.

To release the latching mechanism, the first and second arms 402, 408 are squeezed together. This releases the frictional forces holding the latching plate 418 into place on the hooking member 420. Therefore, the first end of the latching plate 418 is biased into the position shown in FIG. 19, and the hooking member 420 is released from the latching plate 418. The first and second arms 402, 408 are now free to open and close with respect to one another.

The method of using the insertion tool 400 is substantially the same as previously described. In this (and the next) embodiment, however, the shunt and the insertion tool 400 are preferably autoclaved together, rather than separately. To do so, the shunt is placed into the insertion tool, and the handle of the insertion tool 400 is latched shut. At this time, the insertion tool 400 maintains appropriate clearances around the shunt (such as those described above with respect to the first embodiment) so that the shunt may absorb moisture and expand when it is autoclaved without damaging the shunt.

Furthermore, the insertion tool 400 has relatively few components (as few as one in certain implementations), and the components are not under a substantial load, so that the insertion tool 100 is not substantially distorted by the autoclaving process. After the shunt and insertion tool have been sterilized, the shunt is ready for implantation, as described above.

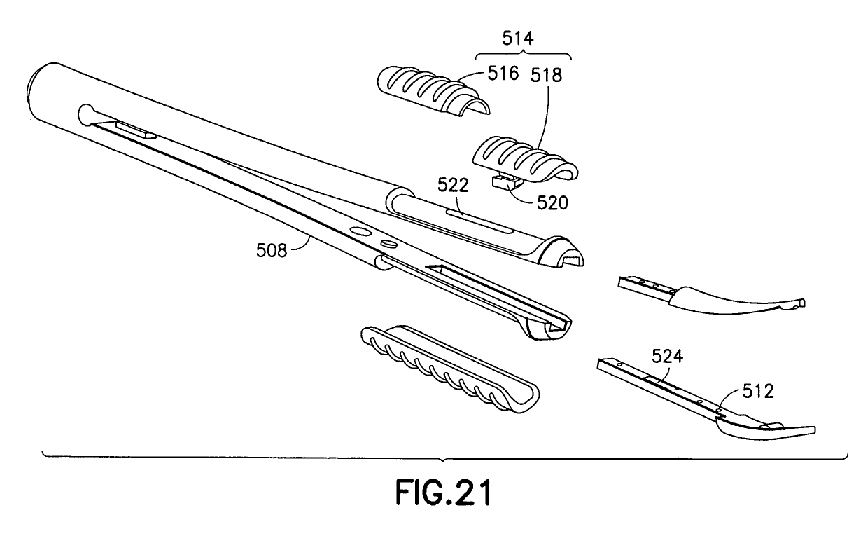

FIGS. 20 and 21 illustrate an insertion tool 500 in accordance with another exemplary embodiment of the present invention. This embodiment of the present invention is substantially similar to the just-described embodiment, except that instead of a “squeeze to release” latching mechanism, this embodiment employs a “slide to release” latching mechanism.

In this embodiment of the invention, the first handle member 514 includes a fixed member 516 and a sliding member 518. The sliding member 518 has a depending hook 520 that passes through a first slot 522 in the first arm 502, and engages a second slot 524 in the distal end 512 of the second arm 508 to hold the first and second arms 504, 508 in a closed position with respect to one another. The depending hook 520 engages the first slot 522 to hold the sliding piece against the first arm 502 in a slidable manner. The sliding member 518 is held in place by a cooperative engagement with the first slot 522. This may be accomplished by making the slot 522 a keyhole slot with the wider portion of the keyhole slot disposed at the proximal end of the first slot 522 (i.e., the end of the slot under the fixed member 516). The sliding member 518 may be inserted into the wider portion of the keyhole slot while the fixed member 516 is removed, and then the fixed member 516 may be replaced to hold the sliding member 518 into the first slot 522.

To close and lock the first and second arms 504, 508 closed with respect to one another, the sliding member 518 is slid into an open position, the arms are closed with respect to one another, and the sliding member is slid into the locked position shown in FIG. 20. The engagement of the hook 520 with the second slot 524 holds the first and second arms 504, 508 closed. To open the first and second arms 504, 508, the sliding member 518 is moved in the opposite direction to release the hook 520 from the second slot 524.

It should be noted that the majority of the components of the exemplary embodiments of the insertion tool illustrated in FIGS. 16-19 and 20-21 are interchangeable. As a result, manufacturing tooling and processes may be shared to minimize manufacturing costs.

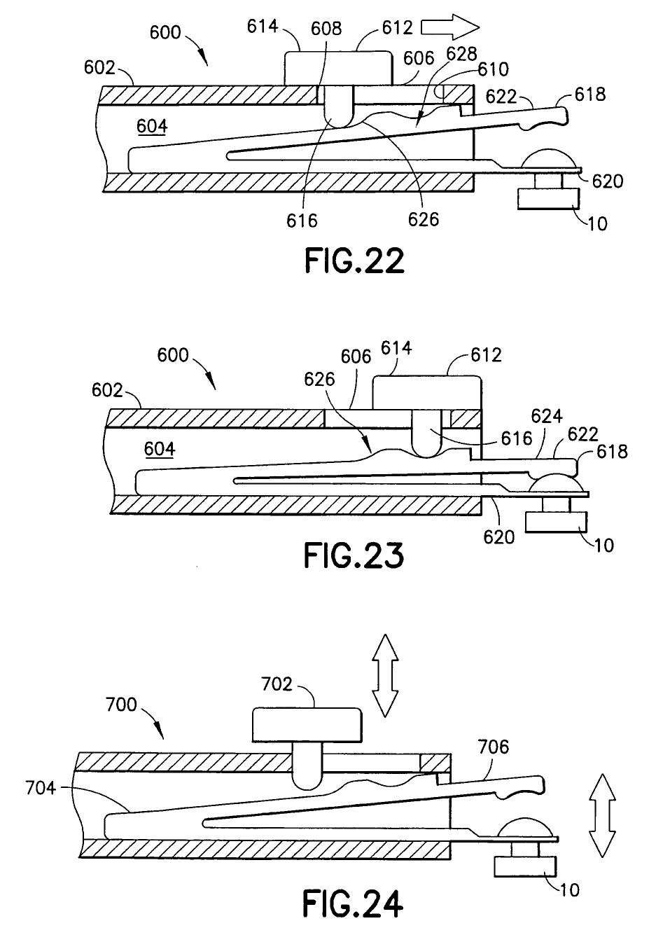

FIGS. 22-23 illustrate a holding device 600 which may be used in conjunction with the insertion tools described above. The holding device 600 includes a hollow, elongated member 602 with an internal cavity 604 which is sized to receive an insertion tool 622. The hollow, elongated member 602 has a slot 606 with a first end 608 and a second end 610. The slot 606 receives a push button slider 612 that may be slid from the first end 608 of the slot 606 to the second end 610 of the slot 606. The push button slider 612 has an outer, handle portion 614, and an inner, depending prong 616. When the push button slider 612 is located at the first end 608 of the slot 606, the inner, depending prong 616 does not touch (or barely touches), the first arm 618 of the insertion tool 622, so that the insertion tool 606 is placed into an open position. In contrast, when the push button slider 612 is located at the second end 610 of the slot 606, the inner prong 616 presses the first arm 618 of the insertion tool 622 downwardly to close the first arm 618 of the insertion tool 622 with respect to the second arm 620 of the insertion tool 622.

To facilitate the movement of the push button slider 612, the depending prong 616 of the slider 612 may be rounded. Also, the upper surface 624 of the first arm 618 may have a rounded cam 626 portion. A detent 628 may be provided on the upper surface of the first arm 618 to receive and engage the depending prong 616 of the slider 612 when the slider is in a closed position. The engagement of the detent 628 and the prong 616 releasably fixes the push button slider 612 (and the insertion tool 622) into a closed position. Although only one detent 628 has been illustrated, it should be understood that multiple detents may be used.

FIG. 24 shows an alternative variation of the insertion tool holder 600 illustrated in FIGS. 22 and 23. In this insertion tool holder 700, the push button slider 702 is allowed to move vertically. The slider 702 may be held in place by any conventional method known to those skilled in the relevant art. Therefore, when the push button slider 702 is in an open position, the push button may be used to open and close the insertion tool 704. Similarly, when the slider 702 is a closed position, the push button slider 702 is biased upward by the first arm 706 of the insertion tool 704. Therefore, the vertical motion allows a surgeon to apply pressure to more firmly force the first arm 706 of the insertion tool 704 toward a closed position.

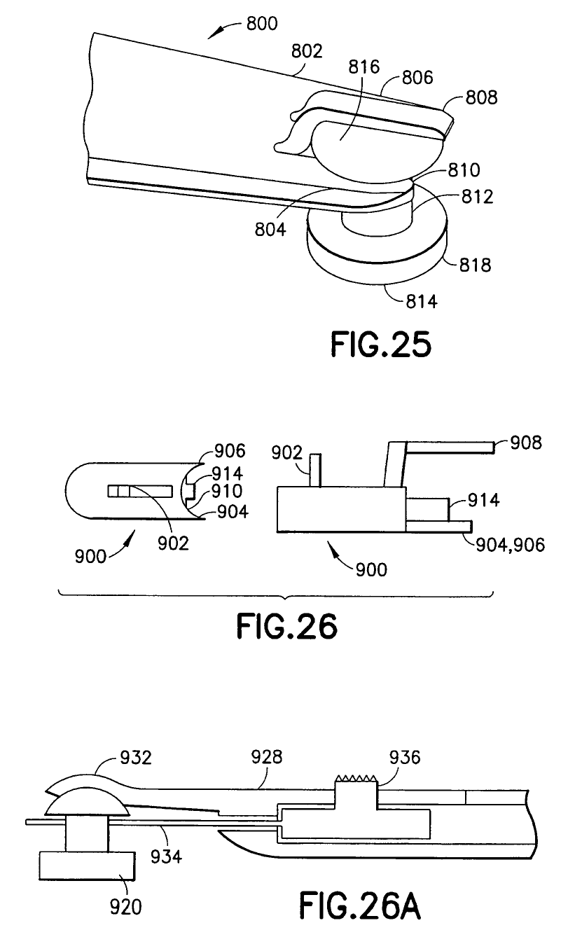

FIG. 25 illustrates the tip of an insertion tool 800 in accordance with another exemplary embodiment of the present invention. In this embodiment of the invention, the tip 802 of the insertion tool 800 includes a blade with three prongs. The first and second prongs 804, 806 form a slot 810. The size of the slot 810 is chosen so that it is large enough to accommodate the body 812 of a shunt 814, but small enough that the head 816 and foot 818 of the shunt 814 cannot pass through the slot 810.

The third prong 808 of the insertion tool 800 gently engages the head 816 of the shunt 814 to hold the shunt in place for implantation. The third prong 808 may be slightly cupped to hold the shunt in place. The third prong 808 may be slightly elastic so that the spring action of the third prong 808 allows for easy release of the shunt after implantation. To assure that the shunt is not dislodged while the insertion tool 800 is being removed, the surgeon may hold the shunt in place while the insertion tool 800 is being withdrawn.

Although not illustrated, the handle of the insertion tool 800 in accordance with this exemplary embodiment of the invention may be similar to other ophthalmic instruments, such as knives. Furthermore, like the above-described embodiments, the tip of the insertion tool is angled so that it minimizes any visual interference and any interference with a patient's anatomy during implantation. The overall length of the tool is such that it may fit into a surgeon's hand, approximately 5″ to 7″ (127 mm to 178 mm), and it may be constructed of a polymer or a metal. It may be manufactured as a single, continuous entity using any suitable method, such as polymer injection molding, MEMS, or wire EDM. Since the insertion tool 800 is a single member, it eliminates the issues caused by numerous parts as described above.

FIG. 26 is a schematic illustration of a variation of the exemplary embodiment of an insertion tool illustrated in FIG. 25. In this variation, the insertion tool 900 has a shunt ejector 914 which is disposed between the first and second prongs 904, 906 and the third prong 908. The shunt ejector 914 is activated by a slider 902. By pushing the slider 902 forward, the shunt ejector 914 is pressed outward, and presses a shunt disposed in the insertion tool 900 out of the slot 910 formed between the first and second prongs 904, 906. The use of an ejection mechanism such as this minimizes any forces on the shunt when the insertion tool is withdrawn after a shunt has been implanted into the cornea.

In the embodiments illustrated in FIGS. 25 and 26, the three prongs of the insertion tool are fixed with respect to one another. In another variation which is illustrated in FIG. 26A, the insertion tool 928 has a third prong 932 that is stationary. The first and second prongs 934 that form a slot for receiving the body of a shunt 920 may be withdrawn, such as by a sliding withdrawal mechanism 936. Thus, once a shunt is implanted, a surgeon may gently withdraw the first and second prongs without moving the insertion tool. At the same time, however, the surgeon may still apply pressure to the head of the shunt so that the shunt remains stable in the desired location. Like the previous embodiment, this minimizes any forces on the shunt caused by the removal of the insertion tool.

As mentioned above, to utilize the insertion tools in accordance with embodiments of the invention described above, the insertion tools must be sterilized, preferably by autoclaving or the like. For the convenience of surgeons, the insertion tools may be sterilized at a manufacturing facility, and then packaged and delivered in a sterilized condition. Furthermore, the shunt may be preloaded onto the insertion tool, and the shunt and insertion tool may be delivered to the surgeon in a single, sterilized package.

The shunt may, however, separate from the insertion tool if it is not properly retained on the insertion tool during transit. Also, if the shunt is not retained on the tool during sterilization procedures, the shunt may fall off the tool during sterilization, or it may fall off the tool when transferred from an assistant to the surgeon.

In the embodiments of the invention shown in FIGS. 15-21, the insertion tool may be locked into a closed position, thereby retaining the shunt on the insertion tool. Similarly, the holder illustrated in FIG. 22 may be used to lock an insertion tool into a closed position to retain a shunt on the insertion tool. Thus, although it is possible to use the retention devices with these embodiments, as currently envisioned, additional retention devices will not be used with these embodiments.

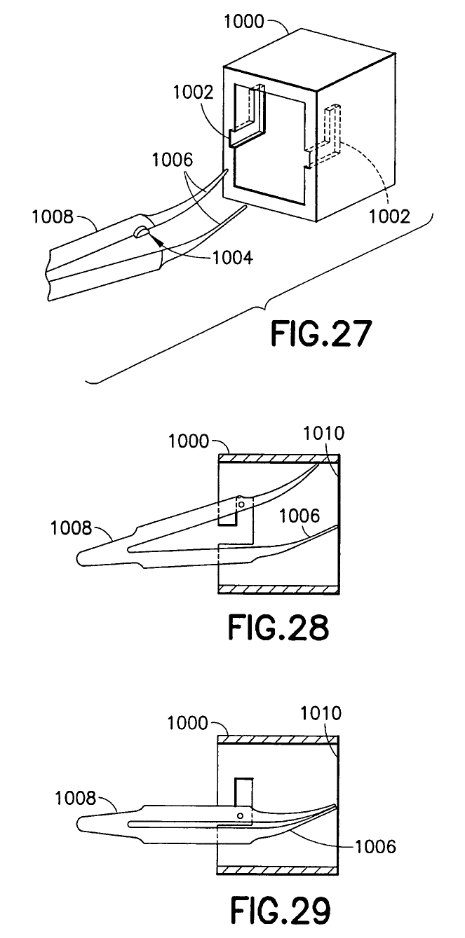

In the embodiments of the invention shown in FIGS. 1-14, however, the shunt must be retained on the insertion tool in another manner. FIGS. 27-34 illustrate various devices for retaining a shunt on an insertion tool. In the device shown in FIGS. 27-29, a cap 1000 is formed to accommodate the distal tips 1006 of the insertion tool 1008. (The shunt is omitted for clarity). The cap 1000 has at least one track 1002, preferably two tracks, that engage engagement pins 1004 on the distal tips 1006 of the insertion tool. The tracks 1002 are L-shaped, so that the engagement pins 1004 of the insertion tool 1002 may be inserted into the tracks 1002 when the insertion tool 1008 is closed. When the engagement pins 1002 reach the crook of the L-shaped tracks 1002, the insertion tool may be released so that it opens. The engagement pins 1004 enter the vertical portions of the L-shaped tracks 1002 so that the cap 1000 is retained on the distal end 1006 of the insertion tool 1008. The inner wall 1010 of the cap 1000 engages the end of the insertion tool 1008 to prevent the shunt from falling off the insertion tool 1008. To remove the cap 1000, the insertion tool 1008 is closed and the cap 1000 is removed, as seen in FIG. 29.

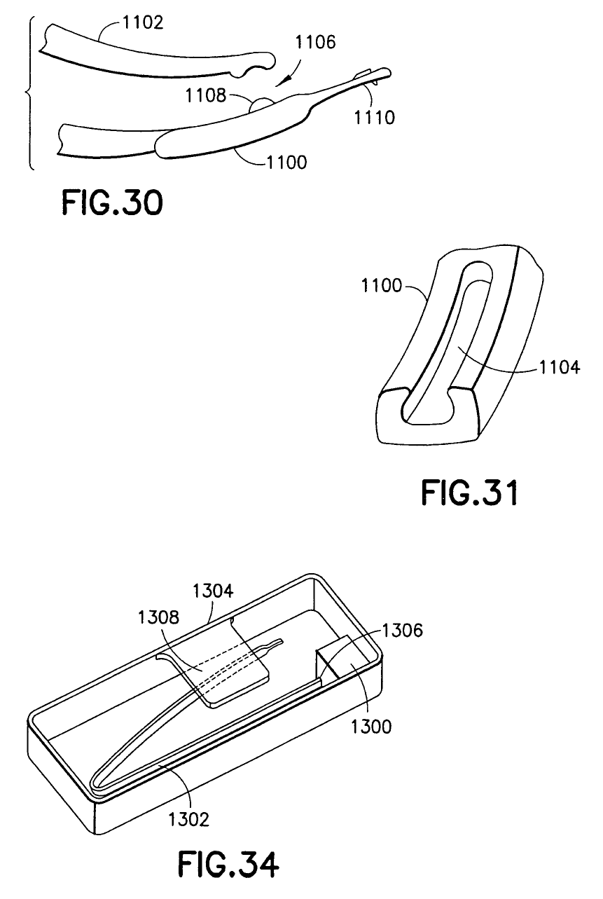

FIGS. 30 and 31 show another variation of a cap 1100 for retaining a shunt on an insertion tool 1102. The cap 1100 is formed of an elastomeric material, and has a slot 1104 which is sized to receive the distal tip 1106 of the insertion tool 1102 that forms a slot for holding the body of a shunt. To retain a shunt on the insertion tool 1102, a shunt 1108 is loaded into the slot in the distal tip 1106 of the insertion tool 1102. Then, the elastomeric cap 1100 is placed over the distal tip 1106 of the insertion tool 1102 to hold the shunt 1108 in place. At this time, since the elastomeric cap 1100 only grasps the edges of the insertion tool 1102, and is open to the top, the shunt 1108 is exposed. This way, the shunt 1108 may be steam sterilized, and may expand without deforming the shunt 1108.

To remove the elastomeric cap 1100 and place the insertion tool 1102 in condition for use, the user grasps the insertion tool 1102 and squeezes the arms of the tool 1102 to close the tool 1102. Since the elastomeric cap 1100 is open and the shunt 1108 is exposed, the arms of the insertion tool 1102 close upon the shunt and retain the shunt on the tool. The user may then peel the elastomeric cap 1100 away from the insertion tool 1102. To facilitate removal of the elastomeric cap 1100, a handle portion 1110 may be provided on the elastomeric cap 1100 to provide a place for the user to grasp the elastomeric cap 1100.

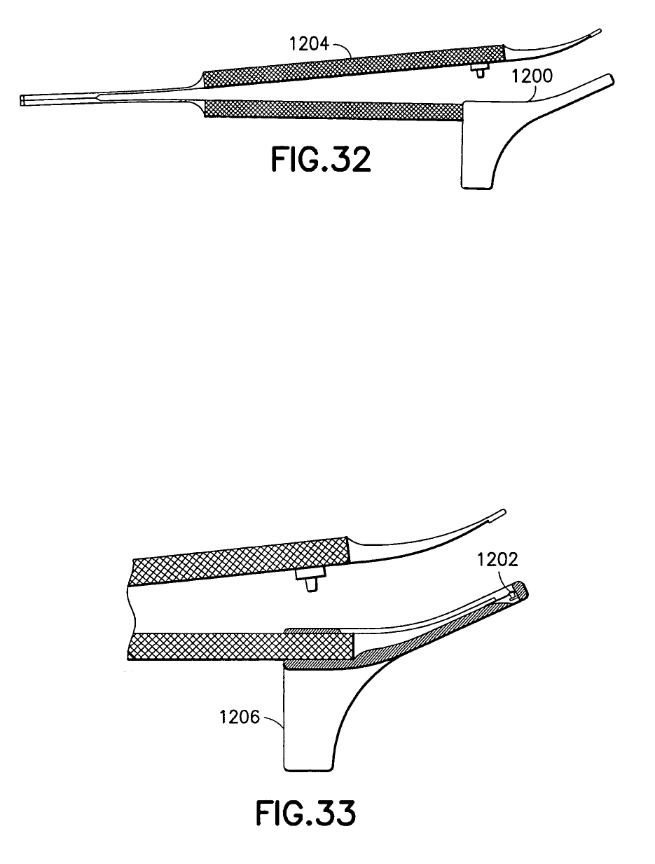

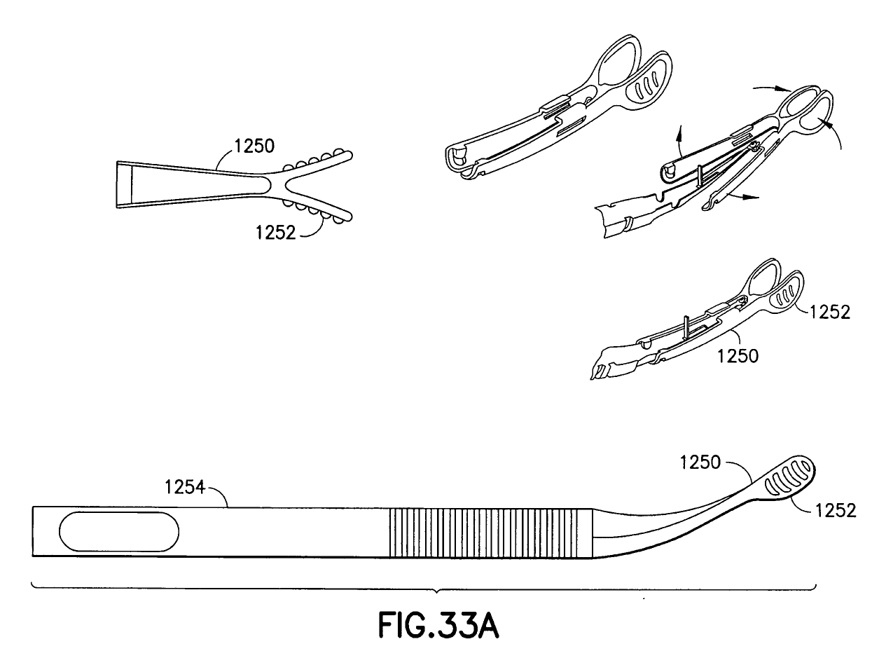

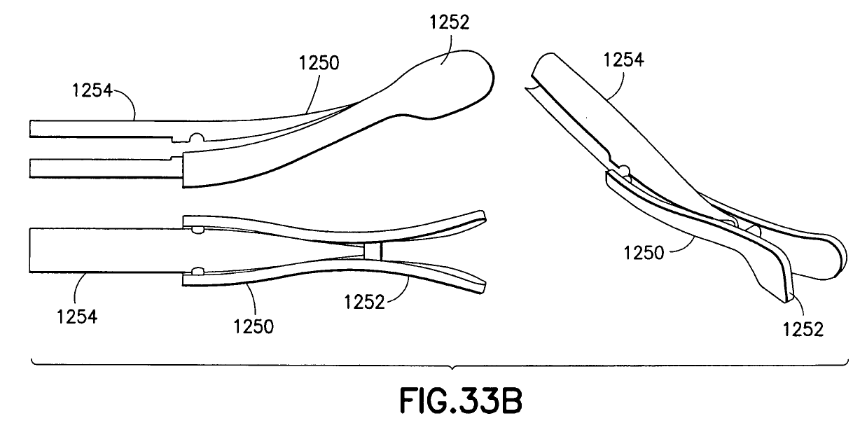

FIGS. 32 and 33 show another variation of an elastomeric cap 1200 for retaining a shunt 1202 on an insertion tool 1204. This variation of the elastomeric cap 1200 is substantially similar to the just-described variation. Here, however, the handle portion 1206 is placed at a lower portion of the elastomeric cap 1200. Other variations of elastomeric caps for retaining the shunt on the insertion tool are also possible.

Although the caps shown in FIGS. 30-33 have been described as elastomeric caps, the caps may also be formed of a relatively rigid material that can be autoclaved, such as polypropylene, polycarbonate, sheet metal, etc. In this case, instead of peeling the cap away, the cap would be slid off the end of the tool to remove the cap. Alternatively, as shown in FIGS. 33A and 33B, the cap 1250 may have arms 1252 which are squeezed together to open the cap 1250 so that it may be removed from the insertion tool 1254.

FIG. 34 shows another variation of a cap 1300 for retaining a shunt on an insertion tool 1302. In this variation, the cap 1300 is formed as an integral part of a package tray 1304 for holding the insertion tool 1302. The tip of the insertion tool 1302 that holds the shunt is placed into a slot 1306. The slot 1306 may be configured to contact the end of the insertion tool so that the shunt is retained on the insertion tool. A retention tab 1308 is provided which holds the insertion tool into the package tray 1304.

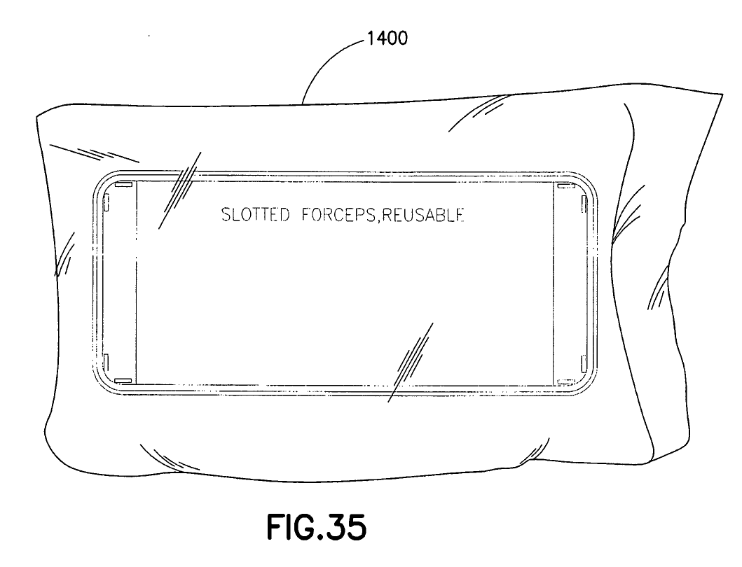

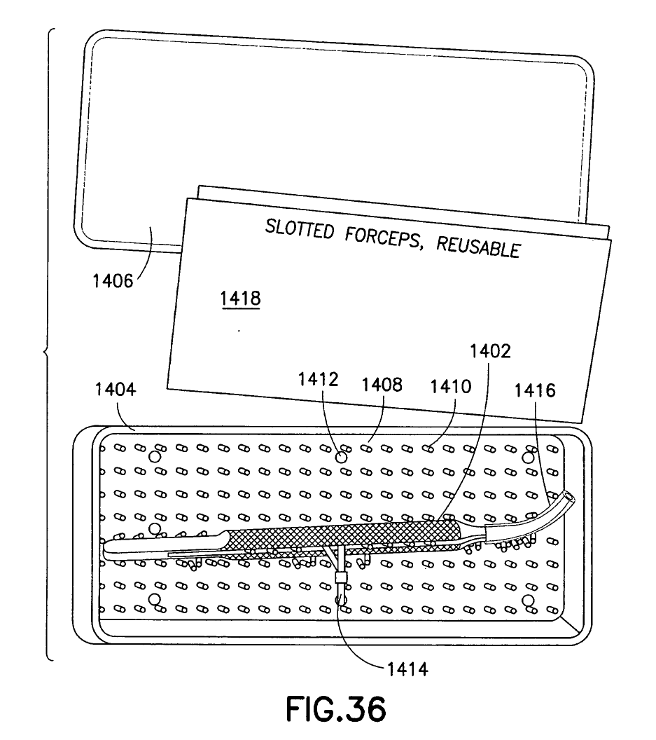

FIGS. 35-36 show another package 1400 for an insertion tool 1402. As seen in FIG. 36, the package 1400 includes a tray 1404 with a lid 1406. An insert 1408 formed of an elastomeric material is disposed on the bottom surface of the tray 1404. The insert 1408 has a plurality of protruding spikes 1410, and a plurality of apertures 1412. To use the package, a shunt insertion tool 1402 is fastened to the insert 1408 by a cable tie 1414 or other suitable rope-like material passed through some of the apertures 1412 in the insert. The protruding spikes 1410 on the insert 1408 engage the shunt insertion tool 1402, and hold the shunt insertion tool 1402 in a relatively stable position. In the illustrated embodiment, the insertion tool 1402 is held into a closed position by a piece of flexible tubing 1416 placed over the tips of the insertion tool 1402.

The lid 1406 of the package 1400 may be transparent so that a packaging insert 1418 with printed information may be viewed through the lid when the lid is closed. Once the insertion tool 1402 is placed in the tray 1404 and the lid 1406 is placed onto the tray, the entire assembly may be placed into a plastic bag, and sealed, as shown in FIG. 35.

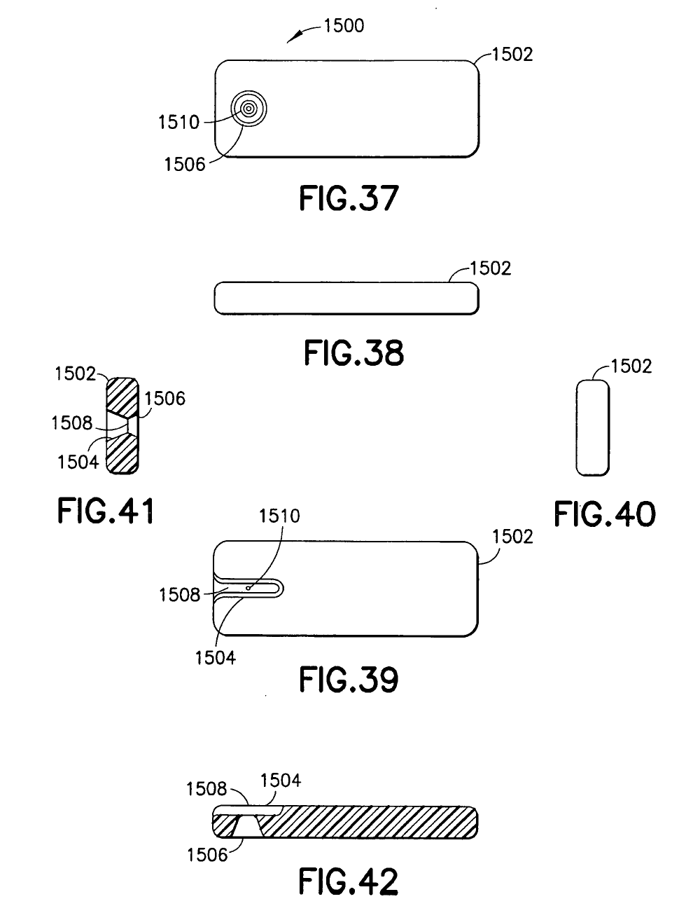

FIGS. 37-42 show a holding device 1500 for a shunt. The holding device 1500 includes a block 1502 formed of an elastomeric material, such as silicone rubber. The block 1502 is preferably proportioned to be easy to hold by a user, and small enough that it may be placed under an operating microscope. Therefore, the surgeon will not need to look away from the operating field to retrieve a shunt from the holding device 1500 during surgery. In the illustrated embodiment, the block 1502 is 0.25″ (6.35 mm) thick.

The block 1502 has a first recess 1504 on one side of the block and a second, opposing recess 1506 on the opposite side of the block 1504. The two opposing recesses form a thin, elastomeric membrane 1508 located between the recesses. The membrane 1508 should be thick enough to hold the shunt securely, yet allow a user to extract the shunt easily. In the illustrated embodiment, the membrane 1508 is approximately 0.007″ thick (0.18 mm). An aperture 1510 is formed in the elastomeric membrane 1508. The aperture 1510 is sized to receive the body of a shunt, but also to allow a shunt to be removed easily, as discussed in further detail below. In the exemplary embodiment, the aperture 1510 is basically a circle with a diameter of 0.032″ (0.81 mm).

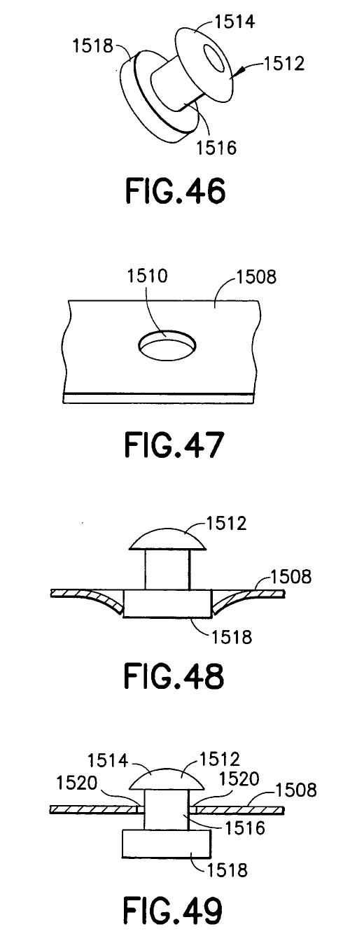

FIGS. 46-51 illustrate the operation of the holding device 1500. As seen in FIG. 46, the shunt 1512 has a spherically domed head 1514, a body 1516, and a foot 1518. As seen in FIG. 47, the membrane 1508 is formed of an elastomeric material with an aperture. To insert the shunt 1512, the shunt 1512 is pressed against the aperture 1510. The membrane 1508 stretches to allow the foot 1518 of the shunt 1512 to pass through (FIG. 48). After the shunt has passed through, the membrane returns to its original state due to the elasticity of the membrane (FIG. 49). Preferably, the shunt is held so that the head of the shunt is located on the top of the holding device. In the embodiment shown in FIG. 49, there are small gaps 1520 between the body 1516 of the shunt 1512 and the membrane 1508 so that the shunt is loosely held. The aperture 1510 may be sized, however, to more firmly grip the shunt without any gaps.

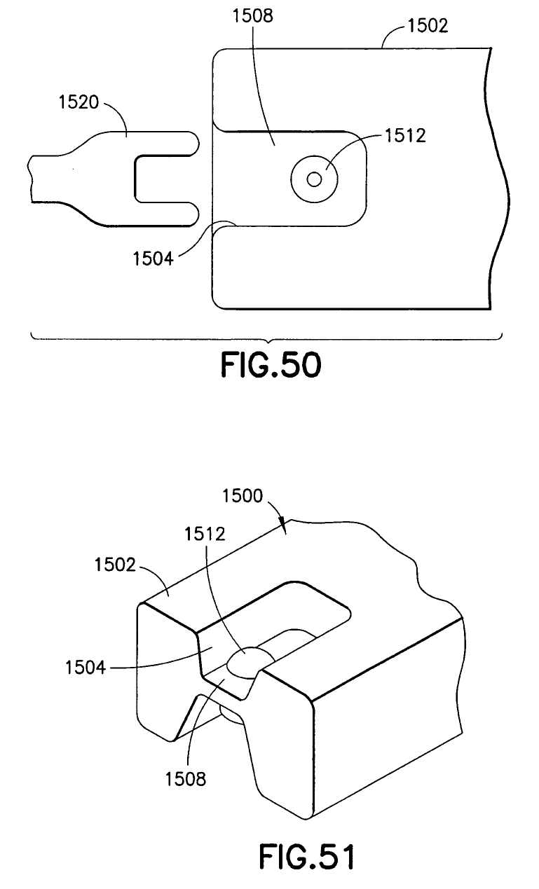

With the shunt 1512 in the position illustrated in FIG. 49, the shunt 1512 may be packaged in a vapor-permeable pouch for sterilization. Since the shunt 1512 is held vertically and with little contact pressure, the shunt 1512 is not distorted during the sterilization process. Furthermore, if the membrane 1508 is formed by recesses 1504, 1506 in a block 1502, as seen in FIG. 51, the shunt is recessed from the surface of the block 1502, and therefore is protected from contact with packaging material or other objects.

To implant the shunt 1512, the surgeon grasps the shunt 1512 with an insertion tool, and gently pulls the shunt 1512 from the membrane. If the membrane 1508 is formed by a recess 1504 in a block 1502, as seen in FIG. 50, a surgeon may slide the tip 1520 of an insertion tool into the recess on the top of the block, close the insertion tool to grasp the shunt 1512, and gently pulls the shunt 1512 to remove the shunt 1512 from the elastomeric membrane.

Preferably, the recess 1504 on the block 1502 is sized to assist the surgeon in locating the shunt 1512 with the insertion tool. In this respect, the recess 1504 forms a slot which is wide enough to receive and guide the tip 1520 of the insertion tool into the proper location to retrieve the shunt 1512.

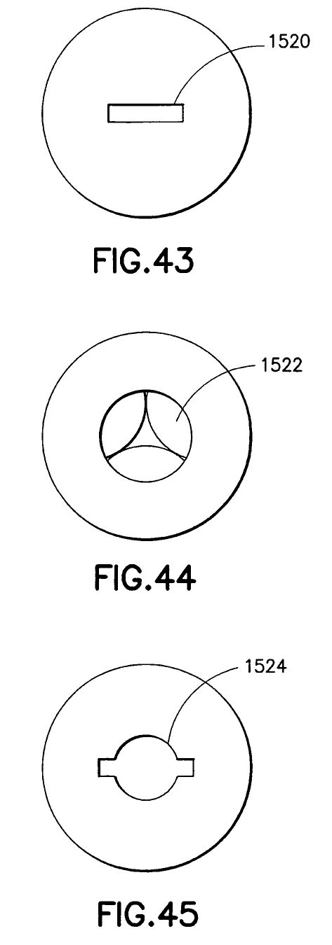

In the illustrated embodiment, the aperture for receiving the shunt 1512 is basically a circular opening. Other apertures may be used, however, such as slots 1520 (FIG. 43), openings with petal-shaped edges 1522 (FIG. 44), and combinations of slots and circular openings 1524 (FIG. 45).

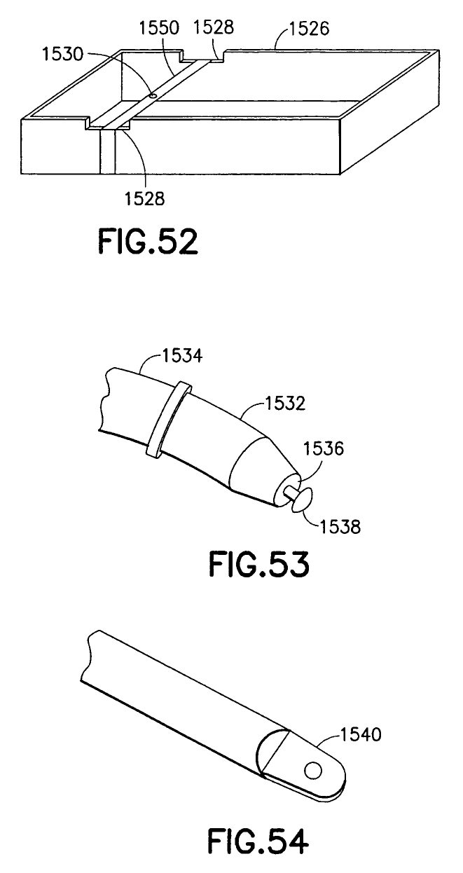

Although so far the elastomeric membrane 1508 has been described with respect to a block of material, the elastomeric membrane may be formed in other configurations. For instance, as illustrated in FIG. 52, the elastomeric membrane may be formed as a simple band of material 1550 with an aperture 1530, and wrapped around a packaging tray 1526, as illustrated in FIG. 52. Preferably, the packaging tray 1526 has recessed portions 1528 for receiving the elastomeric band 1550 so that the shunt is recessed and protected from the packaging material.

The elastomeric membrane may also be formed on the tip of a simple tool which may be placed under the operating microscope. For example, in FIG. 53, an elastomeric cap 1532 is placed over the tip of a tool 1534. The cap 1532 has an elastomeric membrane 1536 formed on end of the cap. The membrane 1536 is substantially perpendicular to the longitudinal axis of the tool 1534 so that the shunt 1538 is held coaxially with the tool. Alternatively, as shown in FIG. 54, the membrane 1540 may be substantially parallel to the longitudinal axis of the tool so that the shunt is held perpendicular to the tool.

The color of the elastomeric material may be chosen so that the shunt is easily visible.

It should be understood that while the above-described devices and methods have been described with respect to ophthalmic shunts, they may be used for other medical devices as well, and the present invention is not limited to ophthalmic shunts. Furthermore, while the above-description specifically refers to transcorneal shunts, it should be understood that the described devices and methods are not specifically limited to transcorneal applications, and may also be used with transscleral and translimbal applications.

While the invention has been shown and described with reference to certain embodiments thereof, it will be understood by those skilled in the art that various changes in form and details may be made therein without departing from the spirit and scope of the invention as defined by the appended claims and their equivalents.

What is claimed is:

1. An insertion tool for implanting an ophthalmic shunt having a foot, a head, and a body connecting the foot and head, the insertion tool comprising:
a first arm having a proximal end and a distal end;
a second arm having a proximal end and a distal end, the second arm being movable with respect to the first arm between an open position and a closed position; and
means for gripping a shunt disposed on the distal ends of the first and second arms.

2. The insertion tool according to claim 1, wherein the gripping means comprises:
a blade with a slot for receiving the body of the ophthalmic shunt disposed on the distal end of the first arm; and
a gripping surface disposed on the distal end of the second arm, the gripping surface facing the blade.

3. The insertion tool according to claim 2, wherein the gripping surface comprises a recess for receiving the head of the ophthalmic shunt.

4. The insertion tool according to claim 3, wherein the gripping surface further comprises an abutment surface for maintaining a gap between the blade and the gripping surface.

5. The insertion tool according to claim 1, wherein the proximal ends of the first and second arm are connected to each other.

6. The insertion tool according to claim 1, further comprising:
an aperture formed in one of the first and second arms; and
an alignment pin extending from the other of the first and second arms, wherein the pin engages the aperture to align the first and second arms.

7. The insertion tool according to claim 6, further comprising
a limit pin for maintaining a gap between the first and second arms.

8. The insertion tool according to claim 7, wherein the gap is adapted to maintain a clearance with a shunt when a shunt is loaded into the tool to allow the shunt to expand.

9. The insertion tool according to claim 7, wherein the limit pin is concentric with the engagement pin.

10. The insertion tool according to claim 1, wherein each arm comprises a handle portion disposed between the proximal and distal ends of the arm.

11. The insertion tool according to claim 10, wherein the handle portion is knurled.

12. The insertion tool according to claim 1, wherein the distal end of each arm portion is curved.

13. The insertion tool according to claim 1, wherein the distal end of each arm is at an angle of approximately 25° with respect to the longitudinal axis of the insertion tool.

14. The insertion tool according to claim 1, wherein the gripping means comprises:
an upper prong and a side jaw fixedly disposed with respect to one another disposed on the distal end of the first arm; and
a moving jaw disposed on the distal end of the second arm, wherein in the closed position the side jaw and moving jaw form a slot for receiving the body of the ophthalmic shunt, and the upper prong accommodates the head of the ophthalmic shunt.

15. The insertion tool according to claim 1, wherein the first and second arms cross over one another, and the first and second arms are normally biased into the closed position.

16. The insertion tool according to claim 1, further comprising:
means for holding the first and second arms in a closed position.

17. The insertion tool according to claim 16, wherein the holding means comprises a latch.

18. The insertion tool according to claim 17, wherein the latch is released by sliding the latch.

19. The insertion tool according to claim 18, wherein the latch comprises:
a hook member;
and a latch member for engaging the hook member.

20. The insertion tool according to claim 17, wherein the latch is released by squeezing the latch.

21. The insertion tool according to claim 20, wherein the latch comprises:
a resilient metal latching plate; and
a hooking member for engaging the resilient metal latching member.

22. The insertion tool according to claim 1, wherein the distal ends of the first and second arms are stamped metal members.

23. The insertion tool according to claim 1, wherein each of the first and second arms is injection molded.

24. The insertion tool according to claim 1, wherein each of the first and second arms are unitary metal arms.

25. The insertion tool according to claim 1, further comprising:
means for opening and closing the first and second arm.

26. The insertion tool according to claim 25, wherein the opening and closing means comprises:
a cam disposed on the second arm, and
a slide for engaging the cam to open and close the second arm.

27. The insertion tool according to claim 26, wherein the cam includes a detent for engaging the slide to latch the first and second arms in a closed position.

28. The insertion tool according to claim 27, wherein the insertion tool is disposed in a hollow enclosure, and the slide is disposed in a slot in the hollow enclosure.

29. The insertion tool according to claim 28, wherein the slide is adapted to move in a first direction to latch the first and second arms in a closed position, and in a second direction substantially orthogonal to the first direction to open and close the jaws.

30. An insertion tool for implanting an ophthalmic shunt having a foot, a head, and a body connecting the foot and head, the insertion tool comprising:
a blade member having first, second, and third prongs, the first and second prongs forming a slot for receiving the body of the shunt, and the third prong for engaging the head of the shunt to hold the shunt into place for implantation.

31. The insertion tool according to claim 30, wherein the third prong comprises an elastic material to release the shunt after implantation.

32. The insertion tool according to claim 30, wherein the three prongs of the insertion tool are fixed with respect to one another.

33. The insertion tool according to claim 30, further comprising a shunt ejector for ejecting the shunt from the insertion tool.

34. The insertion tool according to claim 33, wherein the shunt ejector is disposed between the first and second prongs and the third prong.

35. The insertion tool according to claim 33, further comprising a slider for activating the shunt ejector.

36. The insertion tool according to claim 30, wherein the first and second prongs may be withdrawn with respect to the third prong.

37. A method of implanting an ophthalmic shunt having a foot, a head, and a body connecting the foot and head, the method comprising:
making an incision into the anterior chamber of the eye;
engaging the incision with an edge of the foot of the shunt; and
rotating and displacing the shunt to force the foot of the shunt through the incision and under an inner surface of the cornea.

38. The method according to claim 37, wherein the body of the shunt extends in an axial direction, and the method further comprises gripping the shunt from a lateral direction.

39. The method according to claim 37, wherein the incision is made through the cornea of the eye.

40. The method according to claim 39, wherein the incision is made substantially normal to a surface of the cornea.

41. The method according to claim 39, wherein the incision is oblique to a surface of the cornea.

42. The method according to claim 37, wherein the rotating and displacing movement is induced by rolling a handle of an insertion tool between a surgeon's fingers.

43. The method according to claim 37, further comprising:
determining the thickness of the cornea; and
selecting the location for placement of the shunt in the cornea based on the determination of the thickness of the cornea.

44. The method according to claim 43, further comprising determining the appropriate size of the shunt to be inserted based on the determination of the thickness of the cornea.

45. The method according to claim 37, further comprising:
contacting a surface of the eye with the edge of the foot of the shunt; and
moving the shunt along the surface of the eye until the edge of the foot engages the incision.

46. The method according to claim 37, further comprising applying pressure to the head of the shunt to ensure that the foot of the shunt is passed completely through the cornea.

47. A method of using an insertion tool comprising a first arm having a proximal end and a distal end, a second arm having a proximal end and a distal end, the second arm being movable with respect to the first arm between an open position and a closed position, and means for gripping a shunt disposed on the distal ends of the first and second arms, to insert a shunt into an incision, the method comprising:
closing the first and second arms to grip a shunt with the gripping means;
moving the insertion tool to engage the incision with an edge of the foot of the shunt;
rolling the insertion tool to rotate and displace the shunt to force the foot of the shunt through the incision and under an inner surface of the cornea.

48. The method according to claim 47, wherein the body of the shunt extends in an axial direction, and closing the first and second arms to grip the shunt comprises moving the first and second arms to grip the shunt from a lateral direction.

49. The method according to claim 47, further comprising:
opening the insertion tool to release the shunt from the gripping means; and
withdrawing the insertion tool.

50. The method according to claim 47, wherein the first and second arms are biased towards an opened position, and the operation of opening the insertion tool to release the shunt from the gripping means comprises releasing pressure from one of the first and second arms.

51. The method according to claim 47, wherein the first and second arms are biased towards a closed position, and the operation of opening the insertion tool to release the shunt from the gripping means comprises applying pressure to one of the first and second arms.

52. The method according to claim 47, wherein the operation of opening the insertion tool to release the shunt from the gripping means comprises moving one of the first and second arms in a vertical direction.

53. The method according to claim 47, wherein the operation of opening the insertion tool to release the shunt from the gripping means comprises moving one of the first and second arms in a lateral direction.

54. The method according to claim 47, further comprising holding the arms in a closed position with means for holding the first and second arms in a closed position.

55. The method according to claim 54, further comprising sliding a sliding member to release the means for holding the first and second arms in a closed position so that the insertion tool may be opened.

56. The method according to claim 54, further comprising squeezing the first and second arms to release the means for holding the first and second arms in a closed position so that the insertion tool may be opened.

57. The method according to claim 47, wherein the operation of moving the insertion tool to engage the incision with an edge of the foot of the shunt comprises:
contacting a surface of the eye with the edge of the foot of the shunt; and
moving the shunt along the surface of the eye until the edge of the foot engages the incision.
